(19) United States
(12) Patent Application Publication
(10) Pub. No.: US 2008/0206855 A1
(43) Pub. Date: Aug. 28, 2008

(54) Automated Compound Screening Using Gel-Permeation Matrix and Pin-Based Sample Transfer

(75) Inventors: Daniel G. Sipes, San Diego, CA (US); Mark E. Massari, San Diego, CA (US)

Correspondence Address: GLOBAL PATENT GROUP - KAL;ATTN: MS LAVERN HALL, 10411 Clayton Road, Suite 304, St. Louis, MO, 63131, US

(73) Assignee: KALYPSYS, INC., San Diego, CA (US)

(21) Appl. No.: 11/913,203

(22) PCT Filed: May 5, 2006
(86) PCT No.: PCT/US06/17464

§ 371 Date: Oct. 31, 2007

Related U.S. Application Data
(60) Provisional application No. 60/678,259, filed on May 6, 2005.

(51) Int. Cl. C12M 1/18; G01N 35/10; G01N 33/48

(52) U.S. Cl. 435/287.3; 422/63; 702/19

(57) ABSTRACT

A system for identifying biological activity of one or more samples using prolonged signaling. The system includes a sample plate, an assay in a permeable media, a test plate, an automated liquid handler and an electronic reader. The sample plate includes one or more individual wells which are used for storing one or more samples. A feature of the system uses an automated liquid handler to aspirate samples from the sample plate and to dispense each of these samples directly onto the assay in the permeable media. The automated liquid handler transfers the test plate in communicable range to an electronic reader for reporting transient biological events.

### RELATED APPLICATION(S)

This Application claims priority of U.S. provisional application Ser. No. 60/678,259, filed May 6, 2005

### BACKGROUND OF THE INVENTION

Portions of the disclosure of this patent document may contain material that is subject to copyright protection. The copyright owner has no objection to the facsimile reproduction by anyone of the patent document or the patent disclosure as it appears in the Patent and Trademark Office file or records, but otherwise reserves all copyright rights whatsoever.

### FIELD OF THE INVENTION

The present invention relates generally to the field of high-throughput screening technology. In particular, the invention relates to an automated screening using gel-permeation matrix and pin-based transfer of compounds.

### RELATED ART

Pharmaceutical and biological research methods for screening and identifying agonists of G-protein-coupled receptors (hereinafter “GPCRs”) and activators and inhibitors of activated protein kinase (hereinafter “AMP”) conventionally have used fluorometric imaging plate readers (FLIPR) and microarrayed compound screening (μARCS)-based high-throughput screening formats to detect biological responses in an assay. Specifically, changes in calcium mobilization are detected through the use of calcium sensitive dyes in a high-throughput manner using the FLIPR. Moreover, biological activity and signaling is observed through calcium release. The pharmaceutical and biotechnology industries have searched for GPCR agonists by utilizing such technology to screen test compounds.

Use of FLIPR is well known to those practiced in the art as a format to detect biological responses. For instance, the steps involved in FLIPR includes harvesting cells and placing such in a 384 well format (generally takes a day to complete), changing media to load cells, loading plates on FLIPR, and transferring compounds on a read plate (generally takes another day to complete). The compounds are prepared from a compound source plate, diluted in a buffer, and placed onto an intermediate compound plate.

However, this format is also notoriously slow and unreliable. For example, GPCR screening using FLIPR can take up to two days. The delay can increase costs and delay the availability of data. An issue not addressed by conventional systems is the accommodation of automation in the system to assist in the detection of biological activity.

The μARCS methodology, on the other hand, is an ultra-high-throughput screening platform for the screening of compounds for biochemical activity. It is a well-less screening format where reagents are introduced into the assay by agarose gels. The μARCS methodology provides its ultra high throughput capability by screening compounds that are placed on smooth, homogeneous sheets, such as ChemCards™. ChemCards are the size of a conventional microplate, but can accommodate compound densities up to and above 9,200 compounds per sheet. For instance, 8640 compounds can be arrayed on a microplate-sized sheet of polystyrene. The assay is performed by casting reagents into agarose gel-cards and then applying the gel-cards to the ChemCard, allowing the compounds to diffuse into the agarose medium containing the reagent(s). For instance, the 8640 compounds are assayed by placing reagents cast in agarose gels in contact with the compound sheet. The assay is then imaged using standard imaging techniques.

However, existing technologies such as μARCs require complex sandwich type assay formats that are not readily adaptable to automation. Additionally, μARCS requires resolublization of compounds in order to detect activity.

### SUMMARY OF THE INVENTION

In one or more embodiments, the present invention discloses a system for high throughput screening using a pin-based compound transfer system with an open format agarose gel matrix to identify biological activity of one or more sample compounds.

The system includes a sample plate with one or more individual wells for storing one or more samples; an assay in a permeable media for reporting one or more biological responses; a test plate, which includes a multiwell base covered with a lid, for supporting the permeable media assay; an automated liquid handler having one or more probes for aspirating the samples from the sample plate, and dispensing each of the samples directly into the permeable media assay; and an electronic device for reading and displaying the biological responses.

More specifically, the system includes a robotic arm for positioning the sample plate and/or test plate. The robotic arm is adjustable and has arms and a base member, to provide for selective rotation of the arms in various directions. The probes in the system are dimensioned and arranged for correspondence to the individual wells. Specifically, the process of diffusion through a matrix slows the biological responses to the samples such that corresponding signals can be captured using a conventional plate reader such as the ViewLux. The results pertaining to the location and identity of the biological responses are then digitized.

The samples in the system are selectively moveable relative to its neighboring samples, while the assay can comprise reporter cells expressing a desired GPCR or ion channel. As to the permeable media, it is generally of agarose or methylcellulose, and the transient biological event relates to the perturbation of a G-protein-coupled receptor(s).

The present invention also discloses a methodology for identifying biological activity of one or more samples by transferring in an orderly fashion one or more samples directly onto the surface of a gel-permeation matrix, and determining the location of positive and negative assay results from the gel-permeation matrix and thus the location and identity of specific samples having positive and negative biological activity.

The present invention therefore provides a system and method that will identify biological activity using a process of diffusion that gleans prolonged biological signaling. The present invention also provides a system which effectively accommodates automation and is not only simple to use, but reliable and cost effective to implement. Advantages of the present invention include screening compounds in high concentration, capturing information from the morphology of the signal, and detecting biological activity which is not subject to cell plating variability.

Further features and advantages of the invention, as well as the structure and operation of various embodiments of the invention, are described in detail below with reference to the accompanying drawings.

### BRIEF DESCRIPTION OF THE DRAWINGS

The foregoing and other features and advantages of the invention will be apparent from the following, more particular description of a preferred embodiment of the invention, as illustrated in the accompanying drawings wherein like reference numbers generally indicate identical, functionally similar, and/or structurally similar elements. The left most digits in the corresponding reference number indicate the drawing in which an element first appears.

FIG. 1 is a top view of a sample plate of the present invention;

FIG. 2 is a perspective view a test plate of the present invention;

FIG. 3 is a schematic illustration of an automated liquid handler of the present invention;

FIG. 4 is schematic illustration of a robotic arm of the present invention;

FIG. 5 is another schematic illustration of the automated liquid handler as shown in FIG. 3;

FIG. 6 is an exemplary view showing results of automated compound screening utilizing the present invention; and

FIG. 7 is a block diagram showing a flow chart for the open format compound screening utilizing the present invention.

### DETAILED DESCRIPTION OF AN EXEMPLARY EMBODIMENT OF THE INVENTION

While specific exemplary examples, environments and embodiments for a system of automated compound screening using gel-permeation matrix and pin-based sample transfer are discussed below, it should be understood that this is done for illustration purposes only. A person skilled in the relevant art will recognize that other components and configurations can be used without parting from the spirit and scope of the invention. In fact, after reading the following description, it will become apparent to a person skilled in the relevant art how to implement the invention in alternative examples, environments and embodiments.

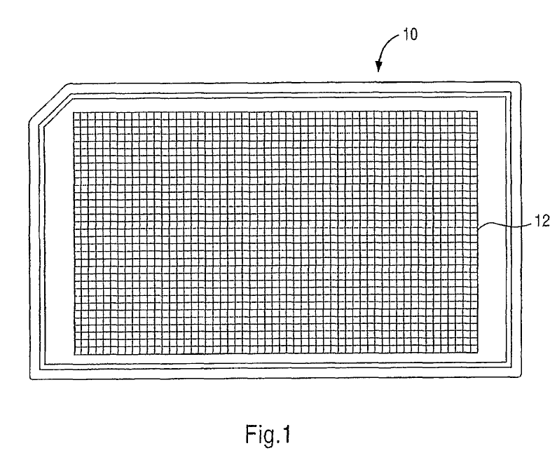

FIG. 1 shows a sample plate 10, which includes one or more individual wells 12 that are used for storing one or more test samples, which can be picoliter in volume and encompass compounds, molecules, cells, cell components, virus, virus components, proteins, etc. The sample plate 10 may include hundreds of test samples located in discrete locations (individual wells) of the plate to be used in a screening process. The number of wells may vary, and each test samples is selectively moveable relative to its neighboring samples. It is noted that two or more test samples can be mixed. For example, a synergy screen with a known compound or drug against a library of compounds, so that library compounds that synergize with the known drug at a desired and perhaps pharmacologically relevant concentration can be identified. An incubator (not shown) is used to store the test samples in the sample plate.

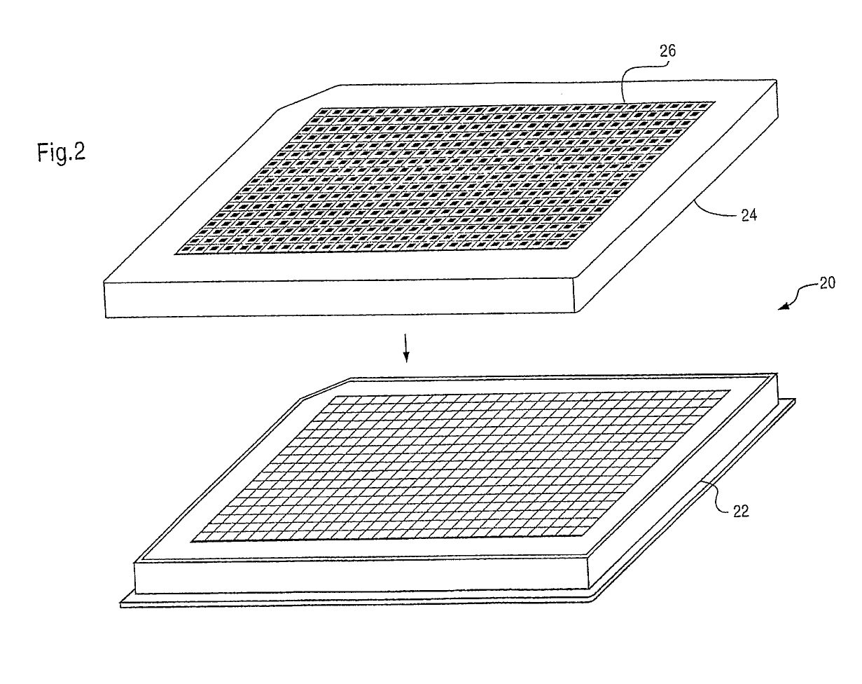

FIG. 2 shows a test plate 20 which has a well base 22 covered with a test lid 24. A gel-permeation matrix (permeable assay matrix) 26, which sets on the test lid 24, can be a homogeneous assay, preferably composed of living cells or biochemical reagents suspended in a mixture of a non-toxic matrix such as agarose and a suitable media or buffer that allows for the detection of transient biological phenomena, such as dye-loaded reporter cells, or a non-cell based biochemical assay, such as an enzymatic assay, prepared in a permeable or porous media (complex gel permeation assay).

The permeable media may be an agar media (e.g., agarose or methylcellulose) that can report transient biological phenomena, such as rapid calcium uptake or release. Other transient biological phenomena that may be reported are ion channels, membrane potential, or other events which are characterized as being “fast” since they occur within seconds. The gel-permeation matrix 26 is used for reporting transient biological events,

In the present embodiment, the gel-permeation matrix 26 is set through an open format (e.g., well-less, gel-permeation format). The gel-permeation matrix 26 is supported by the test plate 20 consisting of the multiwell base 22 and the test lid 24. Various multiwell configurations commonly utilized in the industry, such as the 96-well base plate or the 384-well base plate, can also be applied. The application of the gel-permeation matrix 26 onto the test plate 20 avoids any mixing of agonists or antagonists in the same well, and any undesirable synergistic or additive resulting from compounds in the mixed in the same well.

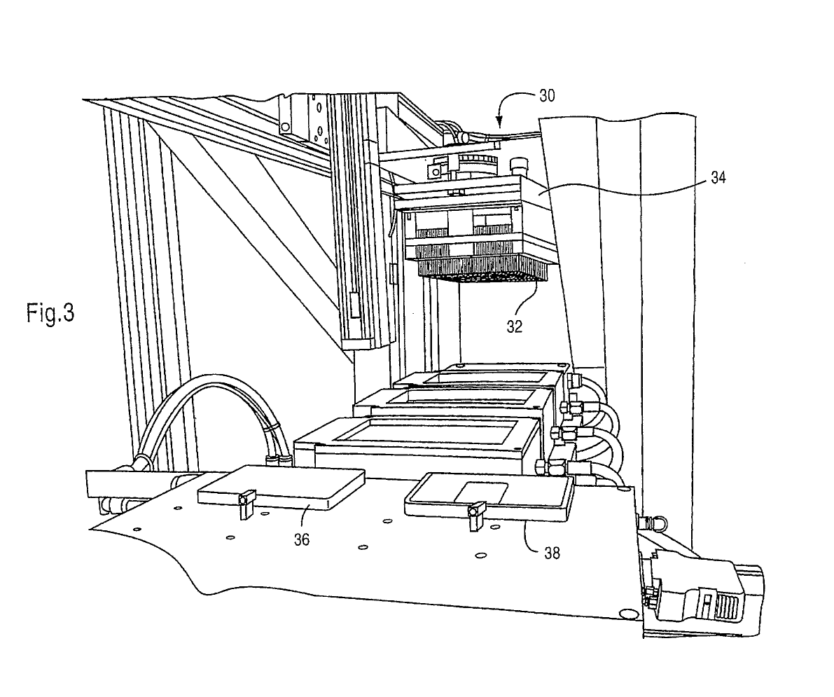

FIG. 3 shows an automated liquid handler 30, which includes a set of probes (pins) 32 mounted on a manipulator arm 34. The manipulator arm 34 is movable in three dimensional space, namely in the X (width), Y (length) and Z (height) directions, with motion based on such parameters as step-function variation and gradient variations. For instance, in one embodiment, the first and second directions are in the same plane, with the third direction being in a plane which is substantially perpendicular to the plane of the third direction, thereby allowing three dimensional motions of probes 32. Computer control device (not shown) effects motion of the manipulator arm 34 through preprogrammed or real-time programmable algorithms.

A sample platform 36 and a test platform are also shown in FIG. 3. The sample platform 36 is used to receive the sample plate 10 from the incubator. In other words, the sample plate from the incubator is transferred onto the sample platform 36 for interaction thereof with the automated liquid handler 30. Once the sample plate 10 is firmly set on the sample platform 36, the automated liquid handler 20 determines the well locations on the sample plate 10 of the samples to be dispensed, then descends one or more probes for retrieving one or more test samples from the sample plate.

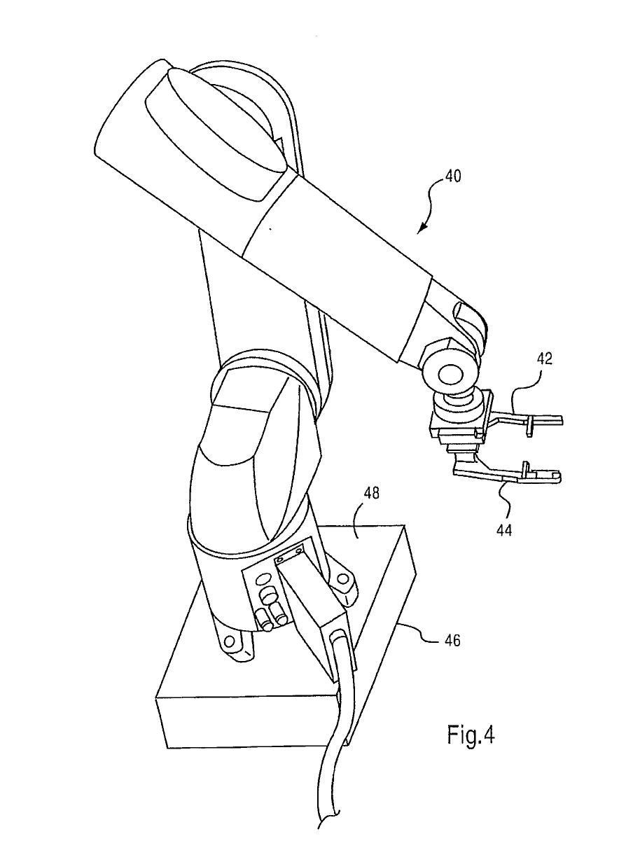

FIG. 4 shows a perspective view of a robotic arm 40, which has two separate arm sections for picking and releasing operations. Preferably, the robotic arm 40 has a left arm section 42 and a right arm section 44 which open and close together. The left arm section 42 is generally synchronized with the right arm section 44 but they can be operated independently of one another. The robotic arm 40 is adjustable, and with arm sections 42 and 44 and a base member 46 having a surface 48, selective rotation of the arms in various directions relative to the surface 48 of the base member can be provided.

One function of the robotic arm 40 is to pick up or release the sample plate 10 containing the test samples to and from the sample platform 36. Another function of the robotic arm 40 is to pick up the test plate 20, which contains the biological activity test data, from the test platform 38 to a location near an electronic readout device, such as a ViewLux machine, to collect and display data. The robotic arm 40 permits aspiration (or dispense) by the probes 32 independently of one another.

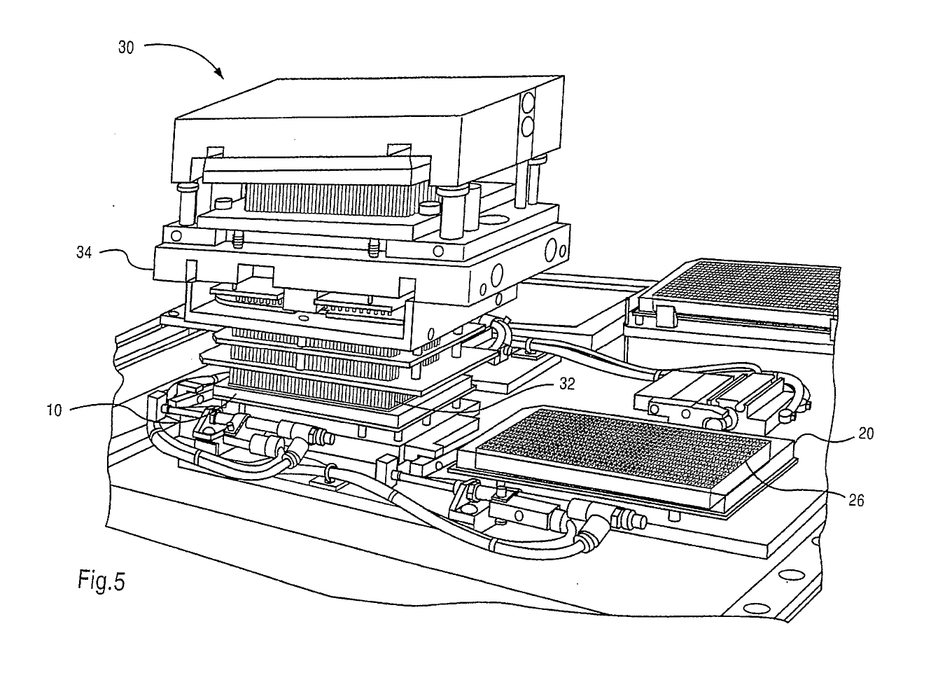

As shown in FIG. 5, the automated liquid handler 30 is used to aspirate samples from the sample plate 10, and to dispense each of the samples directly into the gel-permeation matrix 26. During the aspiration phase of the system, the manipulator 34 descends exemplary probes 32 into respective wells of the sample plate 10, to retrieve or aspirate one or more test samples from the sample plate. Then the automated liquid handler 30 enters a transfer phase to dispense each of the samples to be screened, from in the probes 32 directly into the surface of the gel-permeation matrix 26 on the test plate 20 in an orderly fashion, i.e., predetermined or predefined order. It is noted, however, that the probes 32 can also perform non-contact mediated transfer of test samples to the gel-permeation matrix 26, in conjunction with devices such as Echo and Hummingbird.

In another operation, the automated liquid handler 30 transfers the test plate 20, during the diffusion period or during any biological activity, within communicable range of the electronic detection device such as a fluorescence, luminescence or absorbance-based reader (e.g. ViewLux). Accordingly, when the probes 32 dispense the test samples directly into the gel-permeation matrix 26, sample diffusion begins. The subsequent detection of biological activity can be accomplished through an electronic detection device. A feature of the present invention is the retraction of the probes from the test plate 20.

An aspect of the system is the ability to screen samples over a continuous concentration range. When screening in a plate-based format, each well has a defined volume and therefore the concentration of the sample will be a fixed value for that well. Using the gel-based system, each sample will be tested over a continuous concentration range as it diffuses through the gel permeation matrix 26. Thus, in a single screen a range of concentrations for a given sample will be evaluated without the need to make multiple dilutions. Time and cost savings resulting therefore would be significant.

Another aspect of the system is the ability to screen combinations of different samples for additive, synergistic and or unique biological effects. For example, one sample can be included in the gel matrix at a fixed concentration with the assay components (i.e. cells). Then, another sample or a series of samples can be added to the surface of the gel permeation matrix using the automated liquid handler 30. As a result, one test sample is tested in a concentration range while another test sample is tested at a fixed concentration. The biological readout can then be captured and analyzed for activities that are unique to particular combinations of samples.

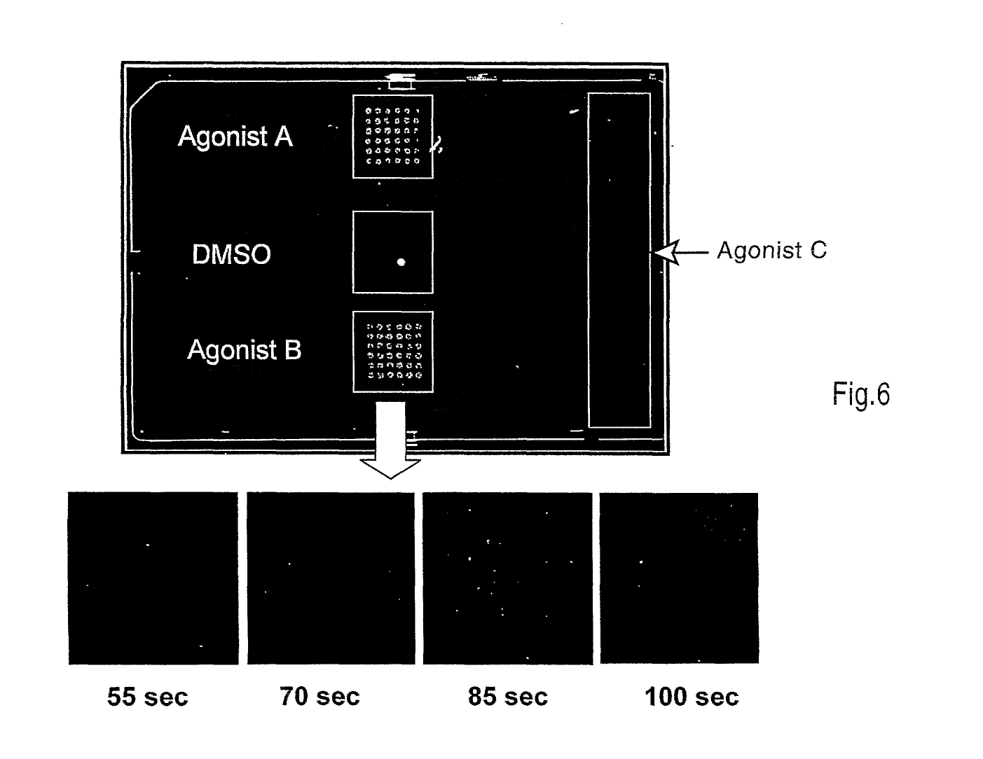

As shown in FIG. 6, the gel image (test plate 20) represents a reporter cell line expressing a known GPCR which has been preloaded with a calcium-sensitive fluorescent dye (e.g. Fluo-4) and imbedded in agarose. Three known agonists of the GPCR, (Agonists A, B and C in FIG. 6) and a negative control (DMSO) have been transferred to the gel matrix 26 by the probes 32. The biological response to the samples, as measured by an increase in fluorescence, is captured and digitized by a ViewLux plate reader. The morphology of the biological response changes in a time-dependent manner as the agonist diffuses through the gel matrix and comes in contact with reporter cells. This is highlighted in FIG. 6, which details the change in signal morphology in 15 second intervals (beginning from 55 sec post sample transfer) in response to Agonist B.

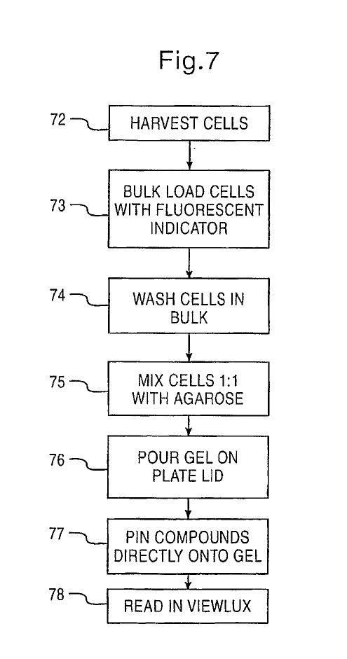

FIG. 7 is a block diagram showing a G-protein-coupled receptor screening in an open format. The steps in the process generally take one (1) day to complete. The open format process begins with cell harvest 72. Next, the load cells are loaded 73 with a fluorescent indicator. The cells are then washed 74 in bulk. Subsequent processing includes a 1:1 mixing 75 of cells with agarose or another appropriate substance, pouring 76 of gel on a plate lid 76, pinning 77 compounds directly onto the gel, and, finally, reading 78 the biological activity in the Viewlux machine.

The location of positive and negative assay results from the gel-permeation matrix 26 can be determined using an imaging device such as CCD or film camera and illumination with suitable wavelengths of light.

Once the location of positive and negative assay results from the gel-permeation matrix 26 are identified, the location and identity of the specific samples having positive or negative biological activity can be identified using a standard laboratory reader or an electronic readout device. Additionally, the relative concentration of the specific samples having positive or negative biological activity can also be determined. Detection of cellular events such as calcium mobilization can be derived from the reader, and thereby enables screening of biological assays with rapid response kinetics such as ion channels and G protein-coupled receptors.

It should be noted that biological activities or events of particular concern, such as perturbation of G-protein-coupled receptors, occur at a fast rate that is within seconds. Other biological activity or events that are also of significance are known as secondary events which occur within minutes to hours. For the measurement of fast events, it is necessary to use specialized equipment with limited throughput.

Accordingly, the present invention allows the process of diffusion to slow until the test samples interact with the receptor. This delay also allows the test plate 20 to be transferred to the electronic reader before the biological response is complete.

The present invention is useful for monitoring changes in intracellular Ca++ levels as well as capturing alterations in membrane potential. Other applications include colony formation assays, neurite outgrowth and assays which require a three-dimensional matrix for growth, survival or differentiation of living cells or tissues.

Another aspect of the invention is for automated high throughput screening of a compound collection against a reporter cell line expressing a GPCR of interest. Several matrices will be evaluated in both open format and multi-well plates for use in high-throughput screening.

The present invention also includes a method for identifying biological activity of one or more samples. The method comprises transferring, through one or more probes and in an orderly fashion, one or more samples (molecules, cells, cell components, virus, virus components, proteins, etc.) directly onto (i.e., on the surface, into the surface and below the surface and any combination thereof) the surface of a gel-permeation matrix; and determining the location of positive and negative assay results from the gel-permeation matrix using an imaging device such as a CCD or film camera and illumination with suitable wavelengths of light, and thus the location and identity of specific samples, which can be picoliter in volume and encompass compounds, having positive and negative biological activity.

Particularly, the transferring step includes aspirating the samples from a sample plate, and dispensing the samples directly onto the gel-permeation matrix (i.e., assay in the permeable media). The determining step includes reading, either kinetically or by end point, transient biological events; and displaying the transient biological events.

Advantages of the open format screening according to the present invention include at least: (1) low cost; (2) allows higher throughput as compared to FLIPR; (3) reliability; (4) accommodates either suspension or adherent cells; (5) allows cell-based or biochemical assays; (6) creates a flexible assay platform; (7) specialized robotics not required; (8) not subject to cell plating variability; (9) information resides in morphology of the signal; (10) uses 100× less compound than FLIPR; and (11) allows screening at high sample concentration and over a continuous range of sample concentrations; and (12) enables screening of combinations of samples.

Skilled persons will recognize the present invention permits a plurality of ways to inventory and track test compound samples, and queue up appropriate sample wells for aspirating/dispensing and detection.

Skilled persons will also understand that the use of any terms throughout the specification depicting particular elements or combinations thereof, are provided by way of example, not limitation, and that the present invention can be utilized and implemented by any systems and methods presently known or possible without escaping from the features and functions disclosed herein.

While various embodiments of the present invention have been described above, it should be understood that they have been presented by way of example only, and not limitation. Thus, the breadth and scope of the present invention should not be limited by any of the above-described exemplary embodiments, but should instead be defined only in accordance with the following claims and their equivalents.

What is claimed is:

1. A system for identifying biological activity of one or more samples, said system comprising:
a sample plate having one or more individual wells for storing one or more samples;
an assay in a permeable media for reporting one or more biological responses to the samples;
a test plate for supporting said permeable media assay;
an automated liquid handler having one or more probes for aspirating said one or more samples from said sample plate, and dispensing each of said samples directly into said permeable media assay; and
an electronic device for reading and displaying the biological responses.

2. The system of claim 1, further comprising one or more robotic arms for positioning said sample plate or said test plate.

3. The system of claim 2, wherein each of said robotic arms is adjustable and has a first arm, a second arm, and a base member having a surface, to provide for selective rotation of said first arm and said second arm in a first direction and in a second direction relative to said surface of said base member.

4. The system of claim 1, wherein said probes are dimensioned and arranged for correspondence to said individual wells.

5. The system of claim 1, wherein a diffusion process slows the biological responses to the samples such that corresponding signals are captured using a plate reader.

6. The system of claim 1, wherein said electronic device digitizes the location and identity of said biological responses.

7. The system of claim 1, wherein each of said samples is selectively moveable relative to its neighboring samples.

8. The system of claim 1, wherein said assay comprises reporter cells.

9. The system of claim 1, wherein the permeable media is agarose or methylcellulose.

10. The system of claim 1, wherein the transient biological event is the perturbation of a G-protein-coupled receptor.

11. The system of claim 1, wherein said test plate is a multi-well plate covered with a lid.

12. The system of claim 11, wherein each of the samples has a predetermined value for interaction with the permeable media assay over a continuous concentration range.

13. The system of claim 11, wherein one of the samples has a predetermined value for interaction with the permeable media assay over a continuous concentration range and another one of the samples is included in the permeable media assay at a fixed concentration.

14. The system of claim 1, wherein said biological responses are transient biological events from perturbation of ion channel activities.

15. The system of claim 1, wherein said permeable media assay is an enzymatic assay.

16. The system of claim 1, wherein said probes allow non-contact mediated transfer of samples to said permeable media assay.

17. A method for identifying biological activity of one or more samples comprising,
transferring in an orderly fashion one or more samples directly onto the surface of a gel-permeation matrix;
determining the location of positive and negative assay results from the gel-permeation matrix and thus the location and identity of specific samples having positive and negative biological activity.

18. The method of claim 15, further comprising determining the relative concentration of two or more specific samples having positive or negative biological activity.

19. The method of claim 15, wherein the transferring step includes aspirating said samples from a sample plate, and dispensing the samples directly onto the gel-permeation matrix.

20. The method of claim 15, wherein the determining step includes reading, either kinetically or by end point, transient biological events; and displaying the transient biological events.
